(19) United States
(12) Patent Application Publication
(10) Pub. No.: US 2012/0197019 A1
(43) Pub. Date: Aug. 2, 2012

(54) COMPOSITIONS AND PROCESSES

(76) Inventors: Dharmesh Surendra Bhanushali, King of Prussia, PA (US); Ted Kiong Chen, King of Prussia, PA (US); Michael A. McGuire, King of Prussia, PA (US); Ravinder Reddy Sudini, King of Prussia, PA (US); Shenyuan Yang, King of Prussia, PA (US)

(21) Appl. No.: 13/500,318

(22) PCT Filed: Oct. 21, 2010
(86) PCT No.: PCT/US2010/053540

§ 371 Date: Apr. 5, 2012

Related U.S. Application Data
(60) Provisional application No. 61/254,303, filed on Oct. 23, 2009.

(51) Int. Cl. C07D 403/12

(52) U.S. Cl. 544/328

(57) ABSTRACT

Methods of making intermediates useful in the synthesis of pazopanib and compositions of such intermediates are described.

### FIELD OF THE INVENTION

The present invention relates to compositions that are useful in the manufacture of pharmaceuticals as well as processes for preparing such compositions.

### BACKGROUND OF THE INVENTION

Pazopanib is a highly bio-available, multi-tyrosine kinase inhibitor of vascular endothelial growth factor receptor (VEGFR)-1, -2, -3, platelet-derived factor receptor (PDGFR)-α, -β, cytokine receptor (cKit), interleukin-2 receptor inducible T-cell kinase (Itk), leukocyte-specific protein tyrosine kinase (Lck), and transmembrane glycoprotein receptor tyrosine kinase (c-Fms). Pazopanib was recently approved by the Food and Drug Administration (FDA) for the treatment of patients with advanced renal cell carcinoma; thus adding to the other FDA-approved VEGF pathway inhibitors, sunitinib, bevacizumab (in combination with interferon) and sorafinib for this same indication.

Processes by which pazopanib and its intermediates can be synthesized have been described in U.S. Pat. No. 7,105,530 as well as in the published PCT application WO03/106416.

### SUMMARY OF THE INVENTION

According to one aspect of the present invention, a composition includes a compound of formula (X):

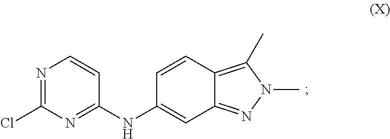

and 0.05-1.0% of a compound of formula (XI):

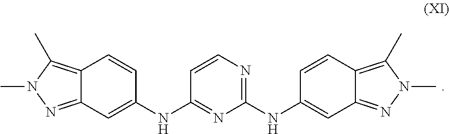

According to another aspect of the present invention, a composition includes a compound of formula (X):

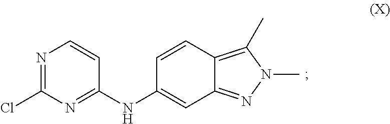

and 1.01-4% of a compound of formula (XI):

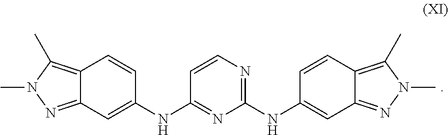

According to still another aspect of the present invention, a process for synthesizing a compound of formula (X):

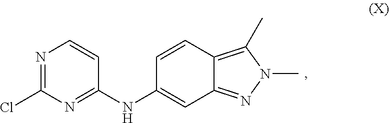

includes combining 2,3-dimethyl-2H-indazol-6-amine and 2,4-dichloropyrimidine in a solvent with sodium bicarbonate having a particle size distribution of >250 μm to provide the compound of formula (X).

According to yet another aspect of the present invention, a process for synthesizing a compound of formula (X):

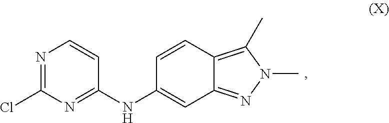

includes combining 2,3-dimethyl-2H-indazol-6-amine and 2,4-dichloropyrimidine in a solvent with sodium bicarbonate having a particle size distribution of 50 μm to 150 μm to provide the compound of formula (X).

According to another aspect of the present invention, a process for synthesizing a compound of formula (X):

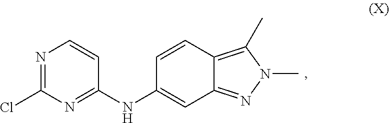

includes combining 2,3-dimethyl-2H-indazol-6-amine and 2,4-dichloropyrimidine in a solvent with a base selected to ensure that the pH of the mixture is less than 7 for no more than 180 minutes to provide the compound of formula (X).

According to still another aspect of the present invention, a process for synthesizing a compound of formula (X):

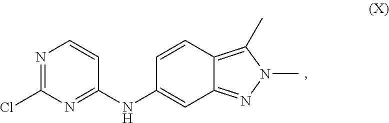

includes combining 2,3-dimethyl-2H-indazol-6-amine and 2,4-dichloropyrimidine in a solvent with a base selected to ensure that the integral of the pH profile over the time that the mixture is less than a pH of 7 is less than 300 to provide the compound of formula (X).

According to yet another aspect of the present invention, a composition includes a compound of formula (XII):

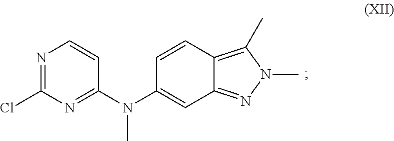

and a total percentage of 0.05-1.0% of compounds of formulae (XIII), (XIV), and/or (XV):

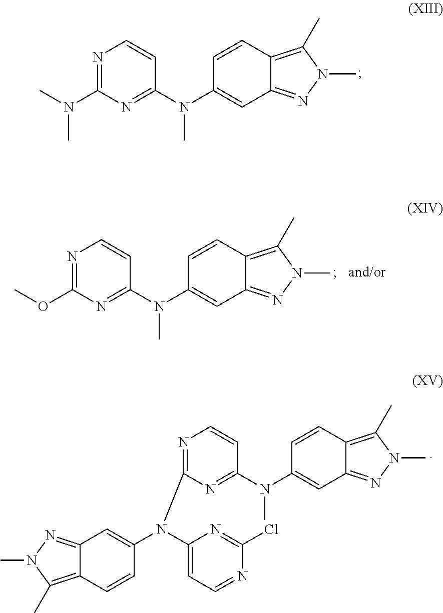

According to another aspect of the present invention, a composition includes a compound of formula (XII):

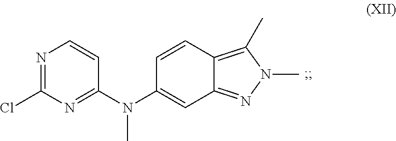

and a total percentage of 1.01-3% of compounds of formulae (XIII), (XIV), and/or (XV):

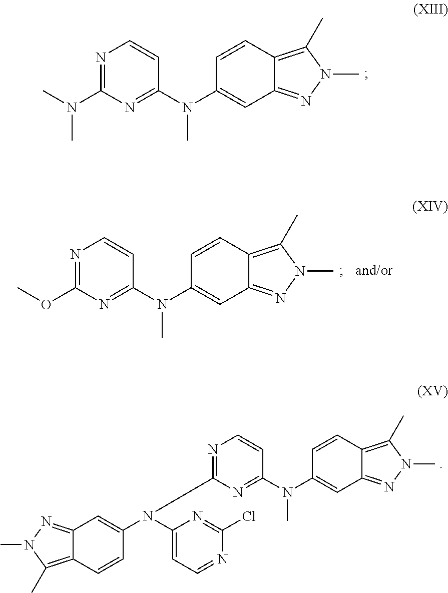

According to still another aspect of the present invention, a process for synthesizing a compound of formula (XII):

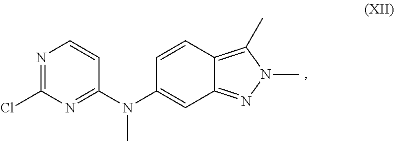

includes suspending N-(2-chloro-4-pyrimidinyl)-2,3-dimethyl-2H-indazol-6-amine and potassium carbonate having a D99 of >300 μm in a solution of an organic solvent and a methylating agent, and heating the reaction mixture to a temperature up to and including reflux for at least 8 hours to provide the compound of formula (XII).

According to yet another aspect of the present invention, a process for synthesizing a compound of formula (XII):

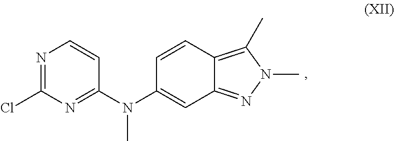

includes suspending N-(2-chloro-4-pyrimidinyl)-2,3-dimethyl-2H-indazol-6-amine and potassium carbonate having a D99 of <200 μm in a solution of an organic solvent and a methylating agent, and heating the reaction mixture to a temperature up to and including reflux for at least 4 hours to provide the compound of formula (XII).

According to another aspect of the present invention, a process for synthesizing a compound of formula (XII):

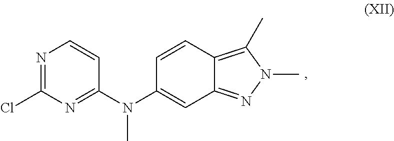

includes combining N-(2-chloro-4-pyrimidinyl)-2,3-dimethyl-2H-indazol-6-amine and a base selected to ensure that the reaction time needed to reduce the level of N-(2-chloro-4-pyrimidinyl)-2,3-dimethyl-2H-indazol-6-amine to less than 2% is less than 8 hours in a solution of an organic solvent and a methylating agent, and heating the reaction mixture to a temperature up to and including reflux for at least 4 hours to provide the compound of formula (XII).

### BRIEF DESCRIPTION OF THE DRAWINGS

FIGS. 1a and 1b illustrate UV and temperature profiles of two reactions in the synthesis of Intermediate 1 heated at different rates; and

FIGS. 2a and 2b illustrate pH and temperature profiles of two reactions run with different particle size distributions of sodium bicarbonate.

### DETAILED DESCRIPTION OF THE INVENTION

The present invention relates to the synthesis of intermediates that are useful in the manufacture of pazopanib. Pazopanib has the following chemical structure:

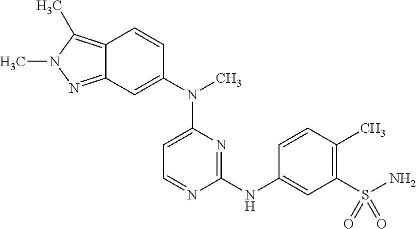

and has the chemical name 5-[[4-[(2,3-dimethyl-2H-indazol-6-yl)methylamino]-2-pyrimidinyl]amino]-2-methylbenzenesulfonamide.

As used herein, the term “between” includes both endpoints unless otherwise stated.

As used herein, the term “D10” means that size in microns below which 10% of the particles lie D[v,0.1].

As used herein, the term “D50” means that size in microns below which 50% of the particles lie D[v,0.5].

As used herein, the term “D90” means that size in microns below which 90% of the particles lie D[v,0.9].

As used herein, the term “D99” means that size in microns below which 99% of the particles lie D[v,0.99].

As used herein, the percentage of a compound in a composition that includes Compound X is expressed as % area as determined by HPLC Method 1 as described herein, unless otherwise specified.

As used herein, the percentage of a compound in a composition that includes Compound XII is expressed as % area as determined by HPLC Method 3 as described herein, unless otherwise specified.

As used herein, particle size distribution is determined by the sieve cut method, which will be understood by those skilled in the art to mean that the smallest diameter sieve on which all particles are retained. For example, a particle size distribution of >250 μm means that a 250 μm sieve is the smallest diameter sieve on which all particles are retained.

According to one aspect of the present invention, a composition includes a compound of formula (X):

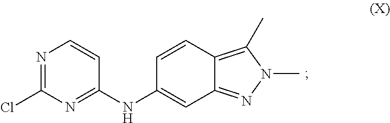

and 0.05-1.0% of a compound of formula (XI):

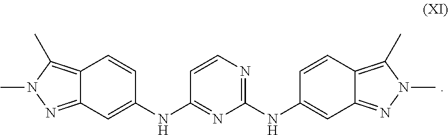

In some embodiments, the amount of the compound of formula (XI) is between a lower limit of 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40% and an upper limit of 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0%.

In some embodiments, the amount of the compound of formula (X) is at least 98.0, 98.1, 98.2, 98.3, 98.4, 98.5, 98.6, 98.7, 98.8, 98.9, or 99.0%.

According to another aspect of the present invention, a composition includes a compound of formula (X):

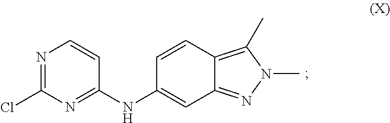

and 1.01-4% of a compound of formula (XI):

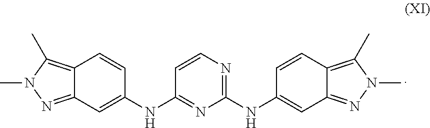

In some embodiments, the amount of the compound of formula (XI) is between a lower limit of 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, or 1.40% and an upper limit of 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0%.

In some embodiments, the amount of the compound of formula (X) is at least 96.0, 96.1, 96.2, 96.3, 96.4, 96.5, 96.6, 96.7, 96.8, 96.9, 97.0, 97.1, 97.2, 97.3, 97.4, 97.5, 97.6, 97.7, 97.8, 97.9 or 98.0%.

According to another aspect of the present invention, a process for synthesizing a compound of formula (X):

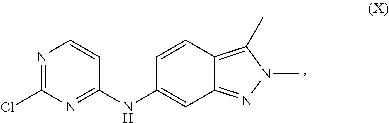

includes combining 2,3-dimethyl-2H-indazol-6-amine and 2,4-dichloropyrimidine in a solvent with sodium bicarbonate having a particle size distribution of >250 μm to provide the compound of formula (X).

According to yet another aspect of the present invention, a process for synthesizing a compound of formula (X):

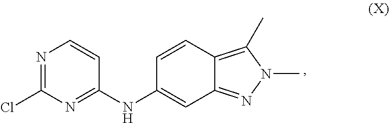

includes combining 2,3-dimethyl-2H-indazol-6-amine and 2,4-dichloropyrimidine in a solvent with sodium bicarbonate having a particle size distribution of 50 μm to 150 μm to provide the compound of formula (X).

In some embodiments, the particle size distribution is between a lower limit of 50, 55, 60, 65, 70, 75, 80, 85, 90, or 95 μm and an upper limit of 90, 95, 100, 105, 110, 115, 120, 125, 130, 135, 140, 145 or 150 μm.

According to still another aspect of the present invention, a process for synthesizing a compound of formula (X):

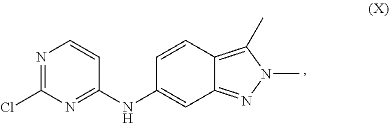

includes combining 2,3-dimethyl-2H-indazol-6-amine and 2,4-dichloropyrimidine in a solvent with a base selected to ensure that the pH of the mixture is less than 7 for no more than 180 minutes to provide the compound of formula (X).

In some embodiments, the base is selected to ensure that the pH of the mixture is less than 7 for no more than 180, 175, 170, 165, 160, 155, 150, 145, 140, 135, 130, 125, 120, 115, or 110 minutes. One of ordinary skill in the art of chemical manufacturing processes can select suitable bases. Exemplary bases include sodium carbonate, sodium hydroxide, barium carbonate, sodium bicarbonate, and di-isopropylethyl amine.

According to another aspect of the present invention, a process for synthesizing a compound of formula (X):

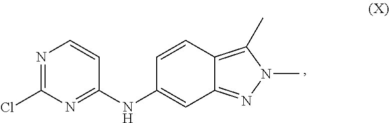

includes combining 2,3-dimethyl-2H-indazol-6-amine and 2,4-dichloropyrimidine in a solvent with a base selected to ensure that the integral of the pH profile over the time that the mixture is less than a pH of 7 is less than 300 to provide the compound of formula (X).

In some embodiments, a base selected to ensure that the integral of the pH profile over the time that the mixture is less than a pH of 7 is less than 300, 275, 250, 225, 200, 175, 150, 125, 100, or 75. One of ordinary skill in the art of chemical manufacturing processes can select suitable bases. Exemplary bases include sodium carbonate, sodium hydroxide, barium carbonate, sodium bicarbonate, and di-isopropylethyl amine.

In embodiments according to the foregoing aspects of the processes for synthesizing compound (X), the solvent is ethanol. In other embodiments, the solvent is industrial methylated spirits ((IMS is a mixture of 95% Ethanol, 4.5% Methanol and 0.5% water). In still other embodiments, the solvent is selected from the group consisting of toluene, DMF, acetonitrile, THF, isopropyl acetate, 1-propanol, ethanol, and 2-ethoxyethanol. As one of skill in the art will appreciate, suitable protic solvents may be used.

In embodiments according to the foregoing aspects of the processes for synthesizing compound (X), the combining is performed at room temperature. As one skill in the art will understand, other temperatures may be used that provide for safe reaction conditions. In some embodiments, the resulting reaction mixture is stirred and heated to reflux for 6, 8, 10 or more hours. In other embodiments, the resulting reaction mixture is stirred and heated to ≧45° C. for 20, 24, 28, 32, 36, 40, 44, 48 or more hours. In some embodiments, the slurry is cooled to 45-55° C. As one of skill in the art will appreciate, other temperatures would work as well. Water is then added to maintain the temperature between 55 and 70° C. The reaction mixture is then stirred at that temperature for 45 minutes to 1.5 hours. The hold time is provided to ensure the product has enough time to crystallize out, and one of skill in the art will appreciate that other hold times will work. The reaction mixture is then cooled to 5-10° C. One of skill in the art will understand that various temperatures such as 25 or 50° C. will work. The mixture is stirred for 45 minutes to 1.5 hours. The hold time is provided to ensure the product has enough time to crystallize out, and one of skill in the art will appreciate that other hold times will work. The product is isolated by filtration and the filter cake is washed with water (2×8.25 L) and ethyl acetate (1×4.95 L). The wet cake is dried under vacuum at 60° C. to provide A compound of formula (X).

According to yet another aspect of the present invention, a composition includes a compound of formula (XII):

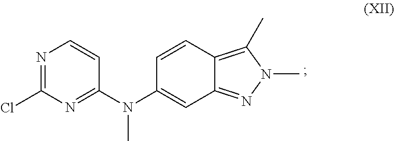

and a total percentage of 0.05-3.0% of compounds of formulae (XIII), (XIV), and/or (XV):

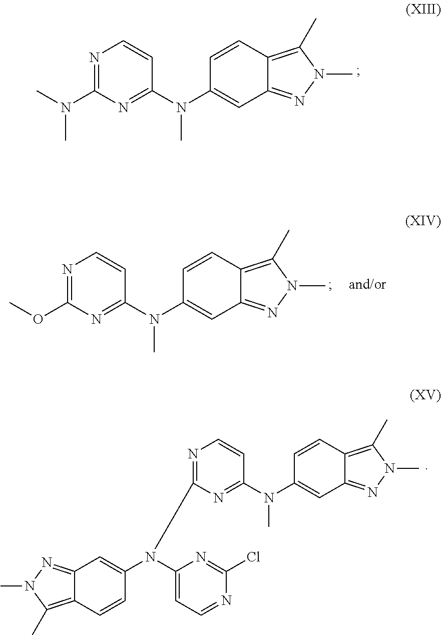

In some embodiments according to this aspect of the present invention, the total amount of the compounds of formulae (XIII), (XIV), and/or (XV) is between a lower limit of 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, or 1.40% and an upper limit of 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9 or 3.0%.

In some embodiments according to this aspect of the present invention, the amount of the compound of formulae (XIII) is between a lower limit of 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40% and an upper limit of 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0%

In some embodiments according to this aspect of the present invention, the amount of the compound of formula (XIV) is between a lower limit of 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40% and an upper limit of 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0%

In some embodiments according to this aspect of the present invention, the amount of the compound of formula (XV) is between a lower limit of 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40% and an upper limit of 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0%

In some embodiments according to this aspect of the present invention, the amount of the compound of formula (XII) is at least 98.0, 98.1, 98.2, 98.3, 98.4, 98.5, 98.6, 98.7, 98.8, 98.9, or 99.0%.

According to still another aspect of the present invention, a process for synthesizing a compound of formula (XII):

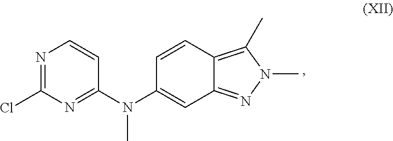

includes suspending N-(2-chloro-4-pyrimidinyl)-2,3-dimethyl-2H-indazol-6-amine and potassium carbonate having a D99 of >300 μm in a solution of an organic solvent and a methylating agent. The reaction mixture is heated to a temperature up to and including reflux for at least 8 hours to provide the compound of formula (XII).

In some embodiments according to this aspect of the present invention, the D99 is >350 μm. In other embodiments, the D99 is >400 μm. In still other embodiments, the D99 is >450 μm. In yet other embodiments, the D99 is >500 μm.

In some embodiments according to this aspect of the present invention, the heating is for at least 8, 9, or 10 hours.

According to yet another aspect of the present invention, a process for synthesizing a compound of formula (XII):

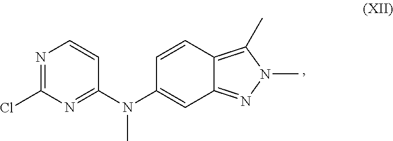

includes suspending N-(2-chloro-4-pyrimidinyl)-2,3-dimethyl-2H-indazol-6-amine and potassium carbonate having a D99 of <200 μm in a solution of an organic solvent and a methylating agent, and heating the reaction mixture to a temperature up to and including reflux for at least 4 hours to provide the compound of formula (XII).

In some embodiments according to this aspect of the present invention, the D99 is <200 μm. In other embodiments, the D99 is <150 μm. In still other embodiments, the D99 is <125 μm. In yet other embodiments, the D99 is <100 μm. In other embodiments, the D99 is <90 μm.

In some embodiments according to this aspect of the present invention, the heating is for at least 4, 5, or 6 hours.

According to another aspect of the present invention, a process for synthesizing a compound of formula (XII):

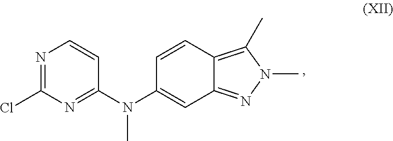

includes combining N-(2-chloro-4-pyrimidinyl)-2,3-dimethyl-2H-indazol-6-amine and a base selected to ensure that the reaction time needed to reduce the level of N-(2-chloro-4-pyrimidinyl)-2,3-dimethyl-2H-indazol-6-amine to less than 2% w/w is less than 8 hours in a solution of organic solvent and a methylating agent, and heating the reaction mixture to a temperature up to and including reflux for at least 4 hours to provide the compound of formula (XII).

In some embodiments, the base is selected to ensure that the reaction time needed to reduce the level of N-(2-chloro-4-pyrimidinyl)-2,3-dimethyl-2H-indazol-6-amine to less than 0.2% w/w is less than 8 hours. In other embodiments, the base is selected to ensure that the reaction time needed to reduce the level of N-(2-chloro-4-pyrimidinyl)-2,3-dimethyl-2H-indazol-6-amine to less than 0.2% w/w is less than 7 hours. Suitable bases include Cs₂CO₃, K₂CO₃, and long straight-chain tertiary amine bases without hindered functioinalities, such as tributyl amine, and triethyl amine.

In some embodiments, the heating is for at least 5 hours. In other embodiments, the heating is for at least 6 hours.

In embodiments according to the foregoing aspects of the processes for synthesizing compound (XII), various organic solvents may be used, including, but not limited to, dimethylformamide (DMF), and high-boiling non-protic solvents such as N-methyl pyrrolidone (NMP), dimethyl acetamide (DMAc), and dimethyl sulfoxide (DMSO). Toluene and 1-butyronitrile are two solvents that do not work well.

In embodiments according to the foregoing aspects of the processes for synthesizing compound (XII), various methylating agents, such as dimethyl carbonate (DMC), N-methyl pyrrolidone, and dimethyl sulfoxide can be used as can various other combos, such as CH₃I/Cs₂CO₃ and TEA/DMC. Dimethyl sulfate and formaldehyde/formic acid will not work for this reaction. In some embodiments, 1, 2, or 3 to 2, 3, 4, or 5 equivalents of potassium carbonate is used. In some embodiments, the methylating agent is used to excess. In other embodiments, 2, 3, or 4 to 4, 5, 6, or 7 equivalents of the methylating agent is used. Equivalents are determined relative to Compound X.

In embodiments according to the foregoing aspects of the processes for synthesizing compound (XII), a nitrogen atmosphere can be used, although this is not required.

In embodiments according to the foregoing aspects of the processes for synthesizing compound (XII),the reaction mixture is heated to a temperature of from 100° C. to reflux.

In embodiments according to the foregoing aspects of the processes for synthesizing compound (XII), the reaction mixture is cooled to less than 105° C. In some embodiments, the reaction mixture is cooled to 55-70° C. Water is charged slowly to the mixture to form a biphasic mixture. The biphasic mixture is stirred for less than 4 hours. In some embodiments, the biphasic mixture is stirred for 1 hour. Then layers are settled for extraction. The bottom aqueous layer is removed to waste. In some embodiments, water is charged over ˜1 hour (though other times can be used) to the organic layer maintaining the reaction temperature between 55-70° C. After addition the mixture is slowly cooled. In some embodiments, the mixture is slowly cooled to 5-10° C., but other temperatures such as 25° C. can be used. The suspension is stirred for 2 hours. In some embodiments, the suspension is stirred at 5-10° C., but other temperatures such as 25° C. can be used. The product is isolated by filtration and then the filter cake is washed with water, followed by cold 1:1 industrial methylated spirits: water. The wet cake is dried under vacuum at 55-60° C. to afford a compound of formula (XII).

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams);
L (liters);
μL (microliters);
M (molar);
N (Normal)
i. v. (intravenous);
MHz (megahertz);
mmol (millimoles);
min (minutes);
mp (melting point);
Tᵣ (retention time);
DCM (dichloromethane);
DMF (N,N-dimethylformamide);
TMSE (2-(trimethylsilyl)ethyl);
TIPS (triisopropylsilyl);
HPLC (high pressure liquid chromatography);
THF (tetrahydrofuran);
EtOAc (ethyl acetate);
EDTA ethylenediaminetetraacetic acid
FBS fetal bovine serum
IMDM Iscove's Modified Dulbecco's medium
PBS phosphate buffered saline
RPMI Roswell Park Memorial Institute
RIPA buffer *
RT room temperature
mg (milligrams);
mL (milliliters);
psi (pounds per square inch);
mM (millimolar);
Kg (kilogram)
Hz (Hertz);
mol (moles);
RT (room termperature);
h (hours);
TLC (thin layer chromatography);
RP (reverse phase);
DCE (dichloroethane);
HOAc (acetic acid);
TMS (trimethylsilyl);
DMSO (dimethylsulfoxide);
DME (1,2-dimethoxyethane);

*150 mM NaCl, 50 mM Tris-HCl, pH 7.5, 0.25% (w/v)-deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

The following examples describe the syntheses of intermediates useful in the synthesis of the compound of formula (I) as well as the synthesis of the compound of formula (I) and the hydrochloride salt and monohydrate monohydrochloride salt thereof:

### Intermediate 1

### Synthesis of Intermediate 1 (N-(2-chloro-4-pyrimidinyl)-2,3-dimethyl-2H-indazol-6-amine) (Intermediate 1)

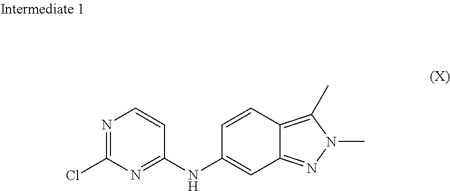

### Procedure 1a

To a solution of 2,3-dimethyl-2H-indazol-6-amine (Starting Material 1 (SM1)) (commercially available from Dottikon Exclusive Synthesis AG, Switzerland and from Divi's Laboratories Limited, India) (1.1 kg, 6.8 mol, 1.0 equiv) and 2,4-dichloropyrimidine (Starting Material 2 (SM2)) (commercially available from Suven Life Sciences Limited, India and from Piramal Healthcare Limited, India) (1.22 kg, 8.1. mol, 1.2 equiv) in 11 L of industrial methylated spirits (IMS is a mixture of 95% Ethanol, 4.5% Methanol and 0.5% water) is charged sodium bicarbonate (>250 μm, sieved, commercially available from Spectrum, located in Gardena, California) (1.26 kg, 15.0 mol, 2.2 equiv) at room temperature. The solution is stirred and heated to reflux for 8 hours. The slurry is cooled to 50° C., and water (5.5 L) is added to maintain the temperature between 55 and 70° C. The reaction is then stirred at that temperature for one hour, and then cooled the reaction mixture to 5-10° C. and stirred for 1 hour. The product is isolated by filtration and the filter cake is washed with water (2×8.25 L) and ethyl acetate (1×4.95 L). The wet cake is dried under vacuum at 60° C. to afford a product that is >97.60% Intermediate 1 (IM1) and 1.08% Impurity 1 by HPLC using the method described below.

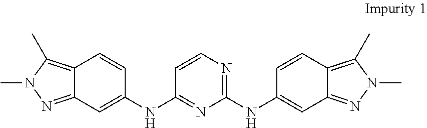

HPLC Method 1

| Analytical Column | 150 mm × 4.6 mm i.d. packed with 3.5 μm | | |
|---|---|---|---|
| | Xbridge C18 or suitably validated alternative | | |
| Mobile Phase A | 10 mM ammonium acetate in water | | |
| Mobile Phase B | Acetonitrile | | |
| Gradient | Time | Mobile | Mobile |
| | (min) | Phase A (%) | Phase B (%) |
| | 0 | 84 | 16 |
| | 20 | 53 | 47 |
| | 20.1 | 84 | 16 |
| | 25 | 84 | 16 |

| | |
|---|---|
| Flow Rate | 1.0 mL per minute |
| Temperature | 40° C. |
| UV Detection Wavelength | 242 nm |
| Sample Concentration | 0.5 mg/mL |
| Injection Volume | 5 μL |

Exemplary relative retention times (RRT) under the chromatographic conditions specified are as follows:

| Compound Number | RRT |
|---|---|
| Intermediate 1 | 1.0 |
| Impurity 1 | 1.2 |

### Procedure 1b

To a solution of 2,3-dimethyl-2H-indazol-6-amine (Starting Material 1 (SM1)) (commercially available from Dottikon Exclusive Synthesis AG, Switzerland and from Divi's Laboratories Limited, India) (1.1 kg, 6.8 mol, 1.0 equiv) and 2,4-dichloropyrimidine (Starting Material 2 (SM2)) (commercially available from Suven Life Sciences Limited, India and from Piramal Healthcare Limited, India) (1.22 kg, 8.1. mol, 1.2 equiv) in 11 L of industrial methylated spirits (IMS is a mixture of 95% Ethanol, 4.5% Methanol and 0.5% water) is charged sodium bicarbonate (>70-105 μm, sieved, commercially available from Glaxo Wellcome Manufacturing, Jurong, Singapore) (1.26 kg, 15.0 mol, 2.2 equiv) at room temperature. The solution is stirred and heated to reflux for 8 hours. The slurry is cooled to 50° C., and water (5.5 L) is added to maintain the temperature between 55 and 70° C. The reaction is then stirred at that temperature for one hour, and then cooled the reaction mixture to 5-10° C. and stirred for 1 hour. The product is isolated by filtration and the filter cake is washed with water (2×8.25 L) and ethyl acetate (1×4.95 L). The wet cake is dried under vacuum at 60° C. to afford a product that is >99.00% Intermediate 1 (IM1) and 0.19% Impurity 1 by HPLC using the method described above in Procedure 1a.

Reaction profiles of procedures similar to procedures 1a and 1b were followed using offline HPLC samples run on the HPLC Method 2 to track Impurity 1 throughout the reaction progress. In addition to offline HPLC measurements of experimental reactions, various process analytical technology instruments (IR, UV, pH) were used.

HPLC Method 2

| Column details (column type, | Zorbax SB-Phenyl, 150 × 4.6 mm, 3.5 | | |
|---|---|---|---|
| particle size and column | μm, or validated equivalent | | |
| dimensions) | | | |
| Column temperature | 40° C. | | |
| Mobile phase A | 0.05% (v/v) TFA in water | | |
| Mobile phase B | 0.05% (v/v) TFA in methanol | | |
| Flow rate | 1.0 mL/min | | |
| Gradient profile | Time (mins) | % A | % B |
| | 0 | 70 | 30 |
| | 30 | 5 | 95 |
| | 30.01 | 70 | 30 |
| | 37 | 70 | 30 |

| | |
|---|---|
| Detector wavelength | 220 nm |
| Injection volume | 8.0 μL |

Exemplary relative retention times (RRT) under the chromatographic conditions specified are as follows:

| Compound Number | RRT |
|---|---|
| Intermediate 1 | 1.0 |
| Impurity 1 | 1.2 |

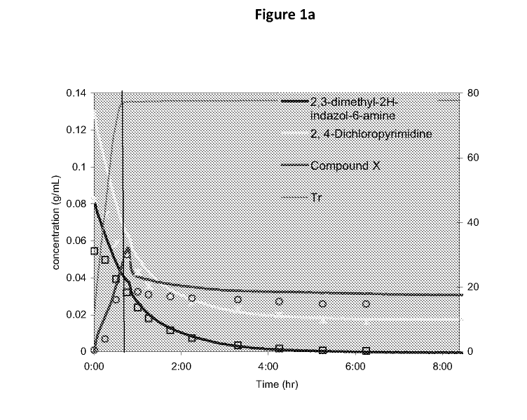

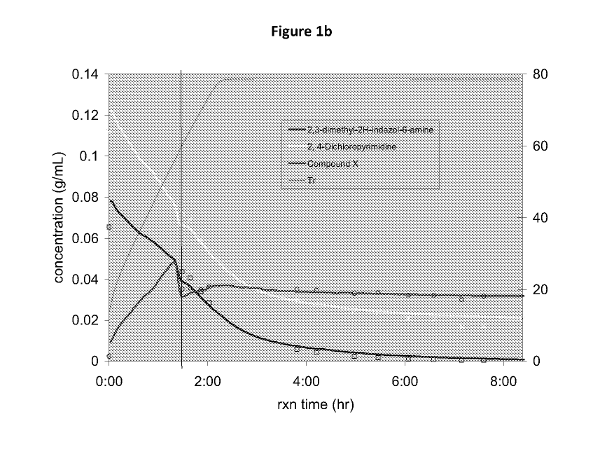

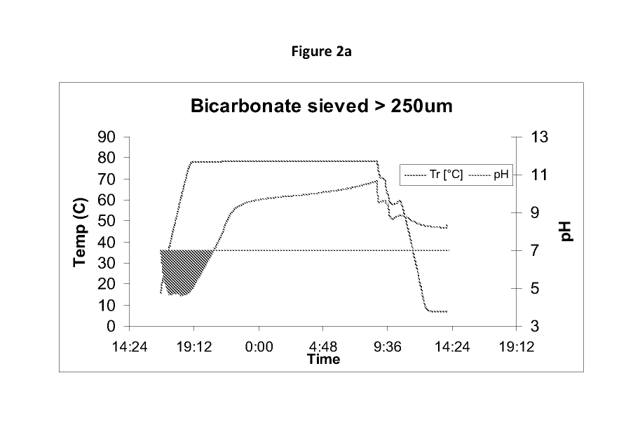

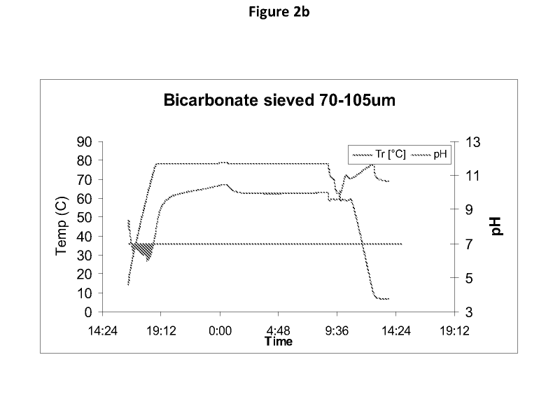

FIGS. 2a and 2b show the pH profile during procedures similar to procedures 1a and 1b. Note that pH drops quickly as the reactor is heated, because HCl is a by-product of the main reaction this profile indicates that the reaction is progressing during heatup. As more evidence of reaction during heatup, FIGS. 1a and 1b show UV overlaid with the temperature profile for two different heating rates; note the significant difference in initial rate of reaction. Note: on FIGS. 1a and 1b, the 2,3-dimethyl-2H-indazole-6-amine curve is indicated by □ and the line that corresponds thereto, the 2,4-dichloropyrimidine curve is indicated by * and the line that corresponds thereto, and the compound X curve is indicated by ∘ and the line that corresponds thereto. The plain curve indicates temperature.

As shown in FIGS. 2a and 2b, the pH levels out in both cases and eventually increases to a basic regime, though for the fine bicarbonate (PSD 70-105 μm), the time and “depth” in the acidic region is much smaller. This indicates that particle size distribution of the base impacts dissolution and further consumption of the acid byproduct of the reaction. This is also an indication that pH is a good way to monitor the reaction progress. In FIGS. 2a and 2b, note that the curve with the shaded portion indicates pH levels while the other curve indicates temperature.

In addition to the variation in the pH profiles, significant differences were seen in the purity of the offloaded Intermediate 1. As shown in Table 1 below, the more acidic the reaction, the more Impurity 1 is present in the product. Also, the higher the value of the integral of the pH profile over the duration of acidic conditions (the shaded regions in FIGS. 2a and 2b), the more Impurity 1 is present in the product.

TABLE 1
Purity results

| | | Sodium | Sodium |
|---|---|---|---|
| Description | | Bicarbonate >250 um | Bicarbonate 75-106 um |
| Isolated IM1 | Area % | 97.60% | 99.00% |
| Isolated | Area % | 1.08% | 0.19% |
| Impurity 1 | | | |
| Minutes | min | 235 | 109 |
| Acidic | | | |
| Integral | min*pH | 387 | 56 |
| Acidic | | | |

### Intermediate 2

### Synthesis of N-(2-chloro-4-pyrimidinyl)-N,2,3-trimethyl-2H-indazol-6-amine (Intermediate 2) and pseudodimer (Impurity 2)

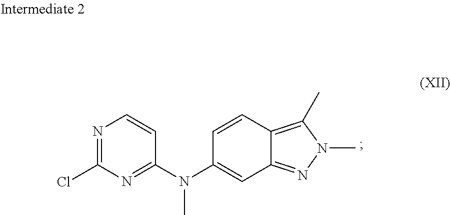

### Procedure 2a

N-(2-chloro-4-pyrimidinyl)-2,3-dimethyl-2H-indazol-6-amine (IM1) (1.31 kg, 4.78 mol, 1 equiv) and potassium carbonate having a D99 as described below of >300 μm (1.96 kg, 14.3 mol, 3 equiv) (commercially available from Armand Products Company, Princeton, N.J.) is suspended in a solution of dimethylformamide (4.3 L) and dimethyl carbonate (2.2 L) under a nitrogen atmosphere. The reaction mixture is heated to reflux (>110° C.) and stirred for 10-12 hours. The reaction mixture is cooled to 55-70° C. and waster is charged (2.6 L) slowly to the mixture. The biphasic mixture is stirred for 1 h and then layers are settled for extraction. The bottom aqueous layer is removed to waste. Water is charged (13.1 L) over ˜1 hr to the organic layer maintaining the reaction temperature between 55-70° C. After addition the mixture is slowly cooled to 5-10° C. and the suspension is stirred for 2 hours at 5-10° C. The product is isolated by filtration and then the filter cake is washed with water (2×5.9 L), followed by cold 1:1 industrial methylated spirits: water (1×2.6 L). The wet cake is dried under vacuum at 55-60° C. to afford a product that is >98% Intermediate 2 (IM2), and <2% combined impurities 2-4 as determined by HPLC and/or LC/MS.

HPLC Method 3

| Column details (column type, | Phenomenex Luna C18(2), 4.6 × 150 | | |
|---|---|---|---|
| particle size and column | mm, 3 μm, or validated equivalent | | |
| dimensions) | | | |
| Column temperature | 40° C. | | |
| Mobile phase A | 0.05% (v/v) TFA in water | | |
| Mobile phase B | 0.05% (v/v) TFA in acetonitrile | | |
| Flow rate | 1.0 mL/minute | | |
| Gradient profile | Time (mins) | % A | % B |
| | 0 | 85 | 15 |
| | 20 | 50 | 50 |
| | 25 | 5 | 95 |
| | 25.01 | 85 | 15 |
| | 30 | 85 | 15 |

| | |
|---|---|
| Detector wavelength | 220 nm |
| Injection volume | 3.0 μL |

Exemplary relative retention times (RRT) under the chromatographic conditions specified are as follows:

| Compound Number | RRT |
|---|---|
| Intermediate 2 | 1.0 |
| Impurity 3 | 0.5 |
| Impurity 2 | 0.6 |
| Impurity 4 | 0.9 |

Impurity 2 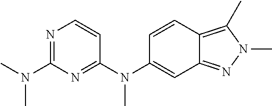
Impurity 3 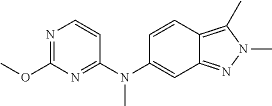
Impurity 4

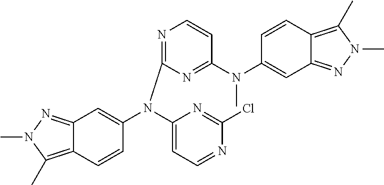

### Procedure 2b

N-(2-chloro-4-pyrimidinyl)-2,3-dimethyl-2H-indazol-6-amine (Intermediate 1) (1.31 kg, 4.78 mol, 1 equiv) and potassium carbonate having a D99 as described below of <100 μM (1.96 kg, 14.3 mol, 3 equiv) (commercially available from Albemarle, Baton Rouge, La.) is suspended in a solution of dimethylformamide (4.3 L) and dimethyl carbonate (2.2 L) under a nitrogen atmosphere. The reaction mixture is heated to reflux (>110° C.) and stirred for 6-7 hours. The reaction mixture is cooled to 55-70° C. and waster is charged (2.6 L) slowly to the mixture. The biphasic mixture is stirred for 1 h and then layers are settled for extraction. The bottom aqueous layer is removed to waste. Water is charged (13.1 L) over ˜1 hr to the organic layer maintaining the reaction temperature between 55-70° C. After addition the mixture is slowly cooled to 5-10° C. and the suspension is stirred for 2 hours at 5-10° C. The product is isolated by filtration and then the filter cake is washed with water (2×5.9 L), followed by cold 1:1 industrial methylated spirits: water (1×2.6 L). The wet cake is dried under vacuum at 55-60° C. to afford a product that is >98% Intermediate 2 (IM2), and <2% combined impurities 2-4 as determined by HPLC and/or LC/MS.

TABLE 2
Potassium Carbonate Particle Size Distribution Analysis (Sympatec)

| | D10 | D50 | D90 | D99 |
|---|---|---|---|---|
| Sigma Aldrich | 7.80 ± 0.16 | 17.96 ± 0.37 | 45.61 ± 2.26 | 144.73 ± 1.19 |
| Jurong (Thasco) | 230.89 ± 1.94 | 355.03 ± 5.43 | 468.38 ± 10.41 | 510.34 ± 1.04 |
| Jurong* (Armand) | 186.65 | 359.81 | 479.74 | 511.47 |
| Albemarle | 3.11 ± 0.36 | 10.34 ± 0.01 | 25.28 ± 0.17 | 84.40 ± 0.19 |

*only one analysis performed

### Intermediate 3

### Synthesis of 5-({4-[(2,3-dimethyl-2H-indazol-6-yl)(methyl)amino]-2-pyrimidinyl}amino)-2-methylbenzenesulfonamide Hydrochloride (Intermediate 3)

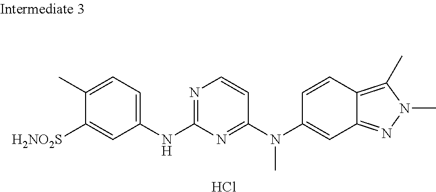

N-(2-chloro-4-pyrimidinyl)-N,2,3-trimethyl-2H-indazol-6-amine (Intermediate 2) (1.66 kg, 5.8 mol, 1.0 equiv) and 5-amino-2-methyl benzenesulfonamide (Starting Material 3 (SM3)) (commercially available from Asymchem Laboratories (Fuxin) Co., Ltd, PR China and from Sumitomo Seika Chemicals Co. Ltd, Japan) (1.19 kg, 6.4 mol, 1.1 equiv) is suspended in 19.9 L of methanol. The mixture is heated to reflux and stirred until complete dissolution is observed. At this stage, the mixture is charged with 4M HCl in 1,4-dioxane (30.0 mL, 0.116 mol, 0.02 equiv) between 60-65° C. The mixture is stirred at reflux for 12 hours. The reaction is deemed complete when the amount IM2 is less than or equal to 0.05% w/w by HPLC. The mixture is cooled to 20-25° C. and stirred for 1 hour. The product is isolated by filtration and the filter cake is washed with acetonitrile (2×5.8 L). The wet cake is dried under vacuum at 50-60° C. to afford Intermediate 3.

### Intermediate 4 and Final Product

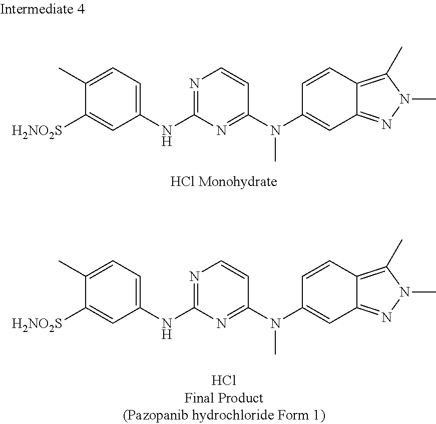

### Synthesis of 5-({4-[(2,3-dimethyl-2H-indazol-6-yl)(methyl)amino]-2-pyrimidinyl}amino)-2-methylbenzenesulfonamide Hydrochloride Monohydrate and 5-({4-[(2,3-dimethyl-2H-indazol-6-yl)(methyl)amino]-2-pyrimidinyl}amino)-2-methylbenzenesulfonamide Hydrochloride Form 1

Intermediate 3 (IM3) (1.4 kg, 2.95 mol, 1.0 equiv) is suspended in 6.1 L of acetonitrile/water, 1.6/1 mixture and is dissolved above 70° C. The clear solution is filtered hot, maintaining the temperature above 55° C. throughout the filtration. The reactor and lines are rinsed with 7.35 L of acetonitrile at 20-60° C. The filtered solution is heated at about 1° C./min to 68-73° C. until dissolved. The clear solution is cooled to 43-47° C. at 0.5 to 1.5° C./min. The solution is held at 43-47° C. for 90 minutes for nucleation of the monohydrate. The monohydrate slurry is cooled to −5 to +5° C. at less than 0.5° C./min. The suspension is held at that temperature (−5 to +5° C.) for up to 36 hours. The product is isolated using a filter dryer with jacket temperature set at to 0° C. The slurry is settled for at least 30 minutes prior to filtration. Mother liquors are removed using 1-2 barg of nitrogen pressure. The cake is washed with 5.6 L of premixed aqueous acetonitrile (75 vol % acetonitrile) at 0-5° C. (held rinse in the reactor until cooled to 0-5° C.). The wash is held on the cake for 30 minutes prior to filtration at 1-2 barg nitrogen pressure. The cake is deliquored at 1-2 barg nitrogen pressure, with the filter dried jacket at 25° C. and the cake is smoothed cake as necessary. After the cake is deliquored, the jacket temperature was increased to 65° C. on the filter drier and vacuum dry the Intermediate 4 (IM4) while agitating the cake. The vacuum is released and the jacket temperature increased to >75° C. with continued agitation until the cake temperature >55° C. is achieved. A separate vessel is charged with 8.4 L of aqueous acetonitrile (3 vol % water) and the solution is heated to >70° C. (28.0 g, 2.5% w/w) micronized Final Product seeds are charged to the form conversion solution. The hot seeded aqueous acetonitrile is transferred to the filter dryer and stirred continuously until XRPD testing indicates that no monohydrate is present and the spectra is concordant with the Form 1 (anhydrate) spectra. After the XRPD indicates for complete Form 1 conversion, the conversion liquors are filtered using 1-2 barg nitrogen pressure. The cake is deliquored with the jacket at 65° C. using 1-2 barg nitrogen pressure and is smoothed as necessary. Vacuum is applied with the jacket at 60° C. and the contents are agitated until the loss on drying (LOD) was obtained <0.5% to afford the Final Product.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1-22. (canceled)

23. A process for synthesizing a compound of formula (X): 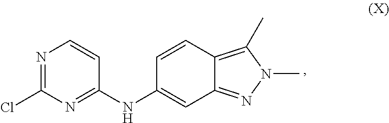
said process comprising:
combining 2,3-dimethyl-2H-indazol-6-amine and 2,4-dichloropyrimidine in a solvent with sodium bicarbonate having a particle size distribution of 50 μm to 150 μm to provide the compound of formula (X).

24. The process according to claim 23, wherein the solvent is ethanol.

25. The process according to claim 23, wherein the solvent is industrial methylated spirits.

26. The process according to claim 23, wherein the particle size distribution is 60 μm to 130 μm.

27. The process according to claim 23, wherein the particle size distribution is 70 μm to 110 μm.

28-64. (canceled)
